(19) United States
(12) Plant Patent Application Publication
(10) Pub. No.: US 2013/0298297 P1
(43) Pub. Date: Nov. 7, 2013

(54) PHK 6 cornus kousa 'Pam's Mountain Bouquet'

(50) ;

(71) Applicants: Robert N. Trigiano, Maryville, TN (US); Phillip A. Wadl, Powell, TN (US); Mark T. Windham, Knoxville, TN (US); Richard M. Evans, Oak Ridge, TN (US)

(72) Inventors: Robert N. Trigiano, Maryville, TN (US); Phillip A. Wadl, Powell, TN (US); Mark T. Windham, Knoxville, TN (US); Richard M. Evans, Oak Ridge, TN (US)

(73) Assignee: UNIVERSITY OF TENNESSEE RESEARCH FOUNDATION, Knoxville, TN (US)

(21) Appl. No.: 13/506,624

(22) Filed: May 3, 2012

(51) Int. Cl. A01H 5/00

(52) U.S. Cl. PLT/220

(57) ABSTRACT

A new and distinct cultivar of flowering dogwood, which has fused bracts is provided. This dogwood is botanically known as Cornus kousa and referred to by the following cultivar name: ‘Pam's Mountain Bouquet’.

### BACKGROUND OF THE INVENTION

The present invention relates to a new and distinct cultivar of flowering dogwood, which has fused bracts. This dogwood is botanically known as Cornus kousa and hereinafter referred to by the following cultivar name: ‘Pam's Mountain Bouquet.

This new dogwood cultivar was discovered in a planting of seedlings at the University of Tennessee (UT) Arboretum and arose from seed gifted to UT by Ms. Polly Hill. ‘Pam's Mountain Bouquet’ is a selection from the original seedlings. Asexual reproduction of ‘Pam's Mountain Bouquet’ by harvested terminal cuttings and grafting of axillary buds onto seedling rootstocks have shown that the unique features of this new dogwood cultivar are stable and reproduced true-to-type in successive generations.

### BRIEF DESCRIPTION OF THE DRAWINGS

FIG. 1. Photograph of ‘Pam's Mountain Bouquet. Colors in the photograph may differ from actual colors due to lighting and light reflectance.

FIGS. 2A and 2B. Close-up Photographs of ‘Pam's Mountain Bouquet” bracts.

FIG. 3. Relatedness of selected Cornus kousa cultivars using 13 SSR markers.

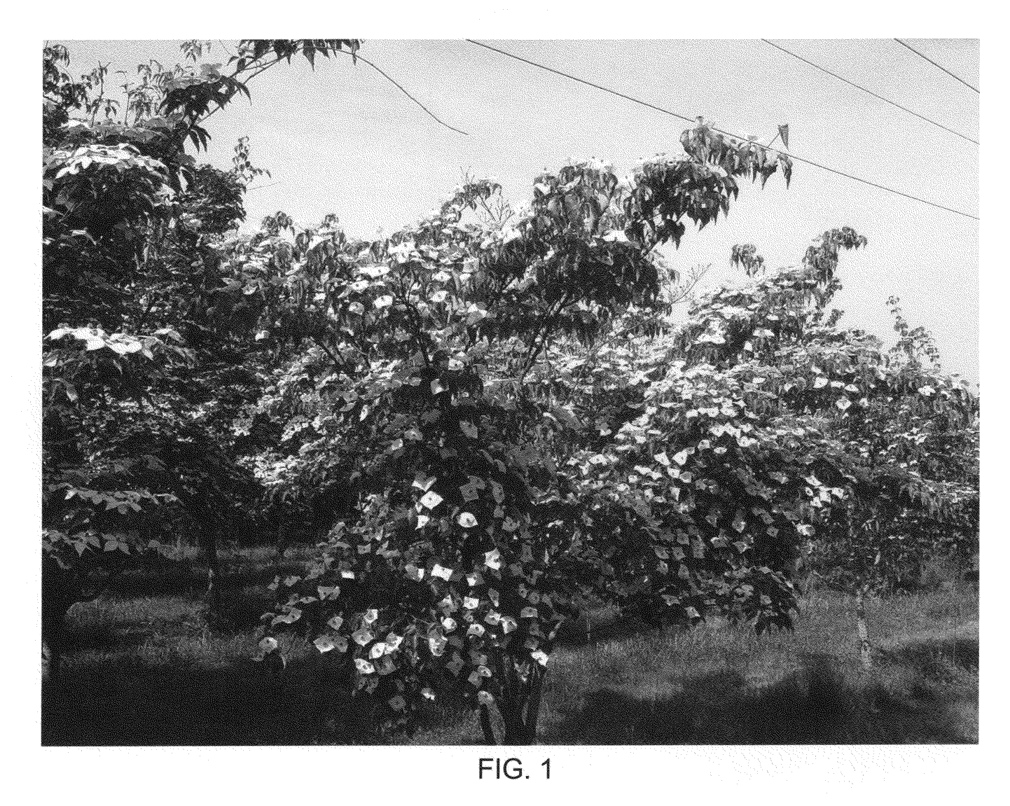

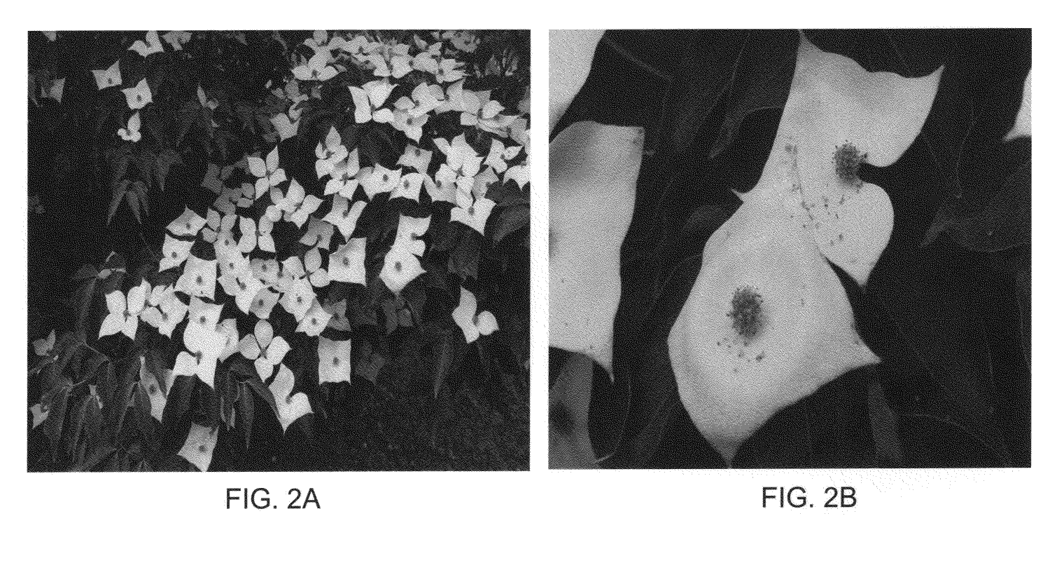

### DETAILED DESCRIPTION OF THE NEW VARIETY

A new and distinct cultivar of flowering dogwood, which has fused bracts is provided. This dogwood is botanically known as Cornus kousa and referred to by the following cultivar name: ‘Pam's Mountain Bouquet’. This cultivar appears to be resistant to powdery mildew and dogwood anthracnose.

This new and distinct dogwood cultivar was discovered in a planting of seedlings at the University of Tennessee (UT) Arboretum in Oakridge, Tenn. and arose from seed gifted to UT by Ms. Polly Hill. ‘Pam's Mountain Bouquet’ is a selection from the original seedlings. Asexual reproduction of ‘Pam's Mountain Bouquet’ by harvested terminal cuttings and grafting of axillary buds onto seedling rootstocks have shown that the unique features of this new dogwood cultivar are stable and reproduced true-to-type in successive generations.

### DETAILED BOTANICAL DESCRIPTION

The following observations, measurements and comparisons describe this cultivar grown in Oak Ridge, Tenn. Dogwoods used for this description were about twenty (20) years old. Plant hardiness is expected to be zones 4-9. The following description uses color references to the Royal Horticultural Society (R.H.S.) Color Chart, except where general terms of ordinary dictionary significance are used. All color ratings were on adaxial surfaces. Measurements are provided as an average (with ranges also provided as indicated).
Botanical classification: Cornus kousa, ‘Pam's Mountain Bouquet’.

Unique Features: This tree features heavy flowering and exhibits fused bracts. About 82% of all bracts on the cultivar exhibit some degree of fusion (one side, two sides or three to four sides being fused; see data in Table 1).

TABLE 1
Cornus kousa ‘Pam’s Mountain Bouquet’ bract characteristics.

| Year | Not fused | One side fused | Two sides fused | 3-4 sides fused |
|---|---|---|---|---|
| 2008 (n = 50) | 7 (14%) | 3 (6%) | 12 (24%) | 28 (56%) |
| 2009 (n = 50) | 10 (20%) | 1 (2%) | 4 (8%) | 35 (70%) |
| 2011 (n = 50) | 9 (18%) | 5 (10%) | 9 (18%) | 27 (54%) |
| Mean | 9 (18%) | 3 (6%) | 8 (16%) | 30 (60%) |

All categories of fused bracts=82%
Color description (based upon Royal Horticultural Society's (R.H.S.) color chart):
   Bark.—198B (greyed-green).
   Leaf.—143B (green).
   Bract.—145A (yellow-green).
   Anther.—N186B (greyed-purple).
   Petal.—145C (yellow-green).
   Peduncle.—144B (yellow-green).
   Peduncle base.—144B (yellow-green).
   Fruit color.—Unripe 143 B; Ripe: 33B to 44A.
Plant description:
Leaf arrangement: opposite
Leaf shape: ovate; tip-acuminate; base-truncate; margin-entire; vestiture-puberulous, recitculate
Canopy: spreading; Height (20 years) 4.9 m (19′6″), width (20 years) 4.7 m (15′ 3″).
Inflorescence: umbel; 82% fused bracts (see data), no petioles; vestiture-puberulous, reticulate; margin-entire; Inflorescence; diagonal 7.4 cm (n=10, range 6.5-8.0 cm); inflorescence length 5.4 cm (n=10, range 4.4- 6.2 cm), inflorescence width, 5.3 cm (n=10, 4.7-5.9 cm); peduncle length 6.8 cm (n=10, range 5.5-8.2 cm); number of individual flowers/inflorescence=34 (n=10, range 27-41). Anther length is a mean of 0.68 mm based on 60 observations.
Disease susceptibility: None noted. Powdery mildew and dogwood anthracnose not observed. Nearby C. florida trees were infested with powdery mildew, but not dogwood anthracnose.
Insect damage: None noted

### REFERENCES

Wadl, P. A., X. Wang, A. N. Trigiano, J. A. Skinner, M. T. Windham, T. A. Rinehart, S. M. Reed, V. R. Pantalone and R N. Trigiano. 2008. Molecular identification key for cultivars and lines of Cornus florida and C. kousa based on microsatellite loci. J. Amer. Soc. Hort. Sci. 133 (6): 783-793.

Wadl, P. W., X. Wang, B. E. Scheffler, T. A. Rinehart, and R. N. Trigiano. 2008. Microsatellites from kousa dogwood (Cornus kousa). Molec. Ecol. Res. 8:780-782. DOI:10.1111/j.1471-8286.2007.0262.x

We claim:

1. A new and distinct cultivar of Dogwood, Cornus kousa, named ‘PAM'S MOUNTAIN BOUQUET’, as illustrated and described.
